(19) United States
(12) Patent Application Publication
(10) Pub. No.: US 2025/0302542 A1
(43) Pub. Date: Oct. 2, 2025

(54) DYNAMIC APPLICATION OF NAVIGATION UPDATES FOR MEDICAL SYSTEMS

(71) Applicant: AURIS HEALTH, INC., Santa Clara, CA (US)

(72) Inventors: Sylvan Seth Canales, San Jose, CA (US); Bridget Branton, Redwood City, CA (US); Daniel Lau, Oakland, CA (US); Jeffrey Adam Kasten, San Mateo, CA (US); Yuqing Pan, Sunnyvale, CA (US); Gang Dong, Redwood City, CA (US); Namita Anil Kumar, Redwood City, CA (US)

(73) Assignee: AURIS HEALTH, INC., Santa Clara, CA (US)

(21) Appl. No.: 18/991,243

(22) Filed: Dec. 20, 2024

Related U.S. Application Data
(60) Provisional application No. 63/571,808, filed on Mar. 29, 2024.

(51) Int. Cl. A61B 34/20; A61B 34/00; A61B 34/10

(52) U.S. Cl. CPC A61B 34/20; A61B 34/10; A61B 34/25; A61B 2034/105; A61B 2034/2065; A61B 2034/254

(57) ABSTRACT

This disclosure provides methods, devices, and systems for planning and performing medical procedures. The present implementations more specifically relate to techniques for navigating an instrument within an anatomy. In some aspects, a controller for a medical system may generate a graphical user interface (GUI) that can be toggled to display a first navigation feature or a second navigation feature based on one or more user inputs, where the first navigation feature depicts a first spatial relationship between the instrument and a target within the anatomy, and the second navigation feature depicts a second spatial relationship between the instrument and the target. In some implementations, the controller may determine the first spatial relationship based on image data captured during a preoperative phase of a medical procedure and may determine the second spatial relationship based on image data captured during an intraoperative phase of the medical procedure.

### CROSS-REFERENCE TO RELATED APPLICATIONS

This application claims priority and benefit under 35 U.S.C. § 119(e) to U.S. Provisional Patent Application No. 63/571,808, filed Mar. 29, 2024, which is incorporated herein by reference in its entirety.

### TECHNICAL FIELD

This disclosure relates generally to medical systems, and specifically to a dynamic application of navigation updates for medical systems.

### DESCRIPTION OF RELATED ART

Many medical procedures include steps that can be performed pre-operation (also referred to as a “preoperative phase”), intra-operation (also referred to as an “intraoperative phase”), or post-operation (also referred to as a “postoperative phase”). For example, during a preoperative phase, an imaging system may be used to scan or otherwise capture images or video of a patient's anatomy. Example suitable imaging technologies include computed tomography (CT), X-ray, fluoroscopy, positron emission tomography (PET), PET-CT, CT angiography, cone beam CT (CBCT), three-dimensional rotational angiography (3DRA), single-photon emission CT (SPECT), magnetic resonance imaging (MRI), optical coherence tomography (OCT), and ultrasound, among other examples. The images may be used, during an intraoperative phase, to help guide or navigate a medical instrument to a target (also referred to as a “treatment site”) within the patient's anatomy. However, images acquired during a preoperative phase may not accurately reflect a spatial relationship between the medical instrument and the target during an intraoperative phase. For example, among various other factors, preoperative images are often acquired several days (or even weeks) before the intraoperative phase, such that changes in the patient's anatomy may cause deviations in the spatial positioning of the target. Thus, there is a need to provide more accurate information about the spatial relationship between the medical instrument and the target during the intraoperative phase.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method of navigating an instrument within an anatomy. The method includes steps of generating a graphical user interface (GUI) that includes a first navigation feature depicting a spatial relationship between the instrument and a target within the anatomy; receiving first image data captured by a first imaging system external to the anatomy while the instrument is disposed within the anatomy; receiving first sensor data via a sensor disposed on the instrument; determining a mapping between a first coordinate space associated with the first imaging system and a second coordinate space associated with the sensor based at least in part on the first image data and the first sensor data; determining an updated spatial relationship between the instrument and the target based at least in part on the mapping between the first coordinate space and the second coordinate space; receiving user input associated with the GUI; and displaying, on the GUI, the first navigation feature or a second navigation feature depicting the updated spatial relationship between the instrument and the target based on the received user input.

Another innovative aspect of the subject matter of this disclosure can be implemented in a controller for a medical system, including a processing system and a memory. The memory stores instructions that, when executed by the processing system, cause the controller to generate a GUI that includes a first navigation feature depicting a spatial relationship between an instrument and a target within an anatomy; receive first image data captured by a first imaging system external to the anatomy while the instrument is disposed within the anatomy; receive first sensor data via a sensor disposed on the instrument; determine a mapping between a first coordinate space associated with the first imaging system and a second coordinate space associated with the sensor based at least in part on the first image data and the first sensor data; determine an updated spatial relationship between the instrument and the target based at least in part on the mapping between the first coordinate space and the second coordinate space; receive user input associated with the GUI; and display, on the GUI, the first navigation feature or a second navigation feature depicting the updated spatial relationship between the instrument and the target based on the received user input.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows example components of the control system and the robotic system of FIG. 1, according to some implementations.

FIG. 3 shows a block diagram of an example localization system, according to some implementations.

FIG. 4 shows a block diagram of an example navigation system, according to some implementations.

FIG. 5 shows another block diagram of an example navigation system, according to some implementations.

FIGS. 6A-6D show various states of an example graphical user interface (GUI) for navigating an instrument to a target within an anatomy, according to some implementations.

FIG. 7 shows a block diagram of an example controller for a medical system, according to some implementations.

FIG. 8 shows an illustrative flowchart depicting an example operation for navigating an instrument within an anatomy, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for case of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many medical procedures include a preoperative phase that precedes an intraoperative phase. During the preoperative phase, for some medical procedures, an imaging system may be used to scan or otherwise capture images or video of at least a portion of a patient's anatomy. For example, a computed tomography (CT) scanner may be used to acquire tomographic images (also referred to as “tomograms” or “CT scans”) of a patient's lungs during the preoperative phase for a bronchoscopy. A tomogram is a cross-section or slice of a three-dimensional (3D) volume. For example, multiple tomograms can be stacked or combined to recreate the 3D volume (such as a 3D model of the patient's lungs). Thus, tomograms can be used to detect a precise location or position (in 3D space) of a nodule or target in the patient's lungs. During the intraoperative phase, for some medical procedures, a medical system may use the preoperative images to generate a graphical interface for navigating a medical instrument within the patient's anatomy. For example, during a bronchoscopy, the medical system may detect a pose of an endoscope (such as a position and orientation of the scope in 3D space) based on sensor data received via an electromagnetic (EM) sensor disposed on the tip of the scope and map the pose of the endoscope to a 3D model of the patient's lungs depicted by the tomograms.

Accordingly, the graphical interface may depict a spatial relationship between the medical instrument and the target within the anatomy based on the sensor data and the image data. However, images acquired during a preoperative phase may not accurately reflect the spatial relationship between the medical instrument and the target during an intraoperative phase. For example, changes in the patient's anatomy or the medical environment can cause the spatial relationship between the endoscope and target to deviate from what is depicted by the graphical interface at any given time, which can lead to inaccurate navigation. Example factors include EM distortion, poor registration (or mapping) between the sensor space and the image space associated with the preoperative scans (also referred to as the “preoperative image space”), outdated preoperative scans, and anatomical deformations, among other examples. Aspects of the present disclosure recognize that some modern imaging technologies (such as cone beam CT) can be used to scan a patient's anatomy during an intraoperative phase. In some aspects, a medical system may capture updated images of the patient's anatomy during the intraoperative phase and use the updated image data to improve the representation of the spatial relationship between the medical instrument and the target. The updated image data and sensor data are often associated with different coordinate spaces. Thus, in some implementations, the medical system may “register” the updated image space with the sensor space to facilitate real-time navigation.

As used herein, the term “registration” refers to a mapping or transformation between different coordinate spaces. For example, a medical system (or registration system associated therewith) may register an imaging system used for capturing images of a patient's anatomy (such as a cone beam CT scanner) with a sensor system used for tracking a pose of a medical instrument within the anatomy (such as an EM field generator) by determining a mapping or spatial transformation that maps any point or vector in the image space to a respective point or vector in the sensor space (such as a transformation matrix). The terms “mapping,” “transformation,” “spatial transformation,” and “registration matrix,” may be used interchangeably herein. The terms “respective” and “corresponding” also may be used interchangeably herein,

Although certain aspects of the present disclosure are described in detail herein in the context of bronchoscopy, it should be understood that the systems and techniques of the present disclosure may be applicable to any medical procedure. Example medical procedures may include minimally invasive procedures (such as laparoscopy), non-invasive procedures (such as endoscopy), therapeutic procedures, diagnostic procedures, percutaneous procedures, and non-percutaneous procedures, among other examples. Example endoscopic procedures include bronchoscopy, ureteroscopy, gastroscopy, nephroscopy, and nephrolithotomy, among other examples. The terms “scope,” “endoscope,” “catheter,” and “instrument” may be used interchangeably herein.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope) to access a target site within a patient's anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a physician). The systems and techniques described herein also may be applicable beyond the context of medical procedures (such as in simulated environments or laboratory settings, such as with models or simulators, among other examples).

Although certain aspects of the present disclosure are described in detail herein in the context of bronchoscopy, it should be understood that the systems and techniques of the present disclosure may be applicable to any medical procedure. Example medical procedures may include minimally invasive procedures (such as laparoscopy), non-invasive procedures (such as endoscopy), therapeutic procedures, diagnostic procedures, percutaneous procedures, and non-percutaneous procedures, among other examples. Example endoscopic procedures include bronchoscopy, ureteroscopy, gastroscopy, nephroscopy, and nephrolithotomy, among other examples.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope) to access a target site within a patient's anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a physician). The systems and techniques described herein also may be applicable beyond the context of medical procedures (such as in simulated environments or laboratory settings, such as with models or simulators, among other examples)

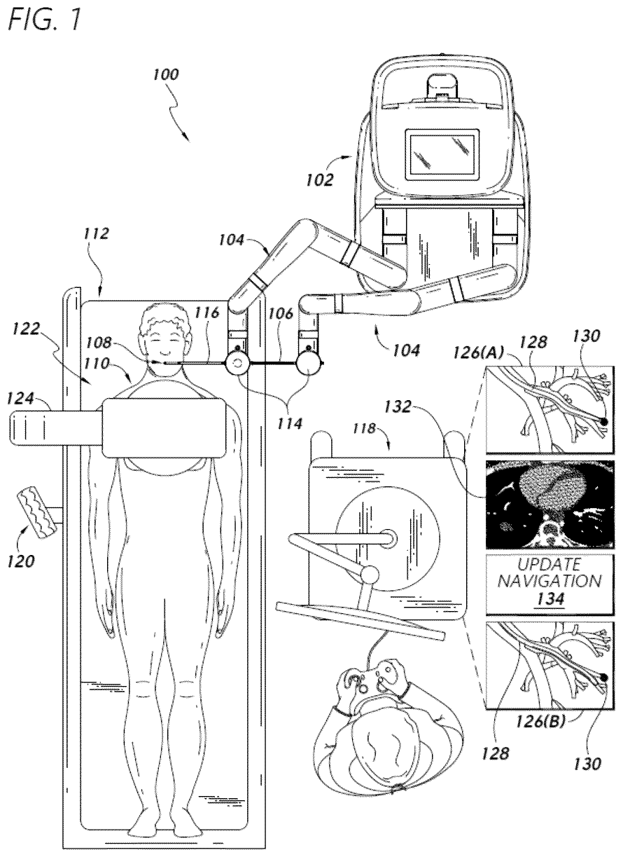

FIG. 1 shows an example medical system 100 (also referred to as a “surgical medical system” or a “robotic medical system”), according to some implementations. As shown in FIG. 1, the medical system 100 may be arranged for diagnostic or therapeutic bronchoscopy. The medical system 100 can include and utilize a robotic system 102 which can be implemented, for example, as a robotic cart. Although the medical system 100 is shown as including various cart-based systems or devices, the concepts disclosed herein can be implemented in any type of robotic system or arrangement, such as robotic systems employing rail-based components, table-based robotic end-effectors, or manipulators, among other examples. The robotic system 102 may include one or more robotic arms 104 (also referred to as “robotic positioners”) configured to position or otherwise manipulate a medical instrument 106 (such as a steerable endoscope or another elongate instrument). For example, the medical instrument 106 can be advanced through a natural orifice access point (such as the mouth 108 of a patient 110 positioned on a table 112) to deliver diagnostic or therapeutic treatment. Although described in the context of a bronchoscopy procedure, the medical system 100 also may be used to perform other types of medical procedures. Example suitable procedures include gastro-intestinal (GI) procedures, renal procedures, urological procedures, and nephrological procedures, among other examples.

With the robotic system 102 properly positioned, the medical instrument 106 can be inserted into the patient 110 robotically, manually, or a combination thereof. For example, the one or more robotic arms 104, or instrument drivers 114 coupled thereto, can control the medical instrument 106. In some implementations, the medical instrument 106 may be advanced within a sheath 116. For example, the sheath 116 may be coupled to, or controlled by, a robotic arm 104. In some implementations, the medical instrument 106 and the sheath 116 may each be coupled to a respective instrument driver from a set of instrument drivers 114. The instrument drivers 114 can be repositionable in space by manipulating the one or more robotic arms 104 into different angles or positions.

In the example of FIG. 1, the medical instrument 106 can be directed down the patient's trachea and lungs after insertion or advanced to a target destination or operative site. In some implementations, to enhance navigation through the patient's lung network or reach the desired target, the medical instrument 106 may be manipulated to telescopically extend from the outer sheath 116 to obtain enhanced articulation or greater bend radius. The use of separate instrument drivers 114 can allow the medical instrument 106 and sheath 116 to be driven independently of each other.

In some implementations, the medical instrument 106 may include an elongate member or shaft configured to be inserted or retracted, articulated, or otherwise moved within the anatomy. Further, in some implementations, the medical instrument 106 may include one or more imaging devices (such as cameras) positioned on a distal end of the elongate shaft or deployed through a working channel of the elongate shaft. The imaging devices can be configured to generate or capture image (or video) data or send the image data to another device or component. In some implementations, the medical instrument 106 may include an instrument base or one or more handles positioned at a proximal end of the medical instrument 106. The instrument base can be coupled to a manipulator (such as an end of a robotic arm 104). The instrument base can include one or more drive inputs coupled to one or more drive outputs of the manipulator, wherein the drive inputs or drive outputs act as an interface.

In some implementations, the medical instrument 106 may include a working channel configured to receive one or more other instruments or elements therein or provide other functionality. The working channel can extend axially, such as along the length of the medical instrument 106. Furthermore, the medical instrument 106 can include or be associated with one or more elongate movement members (such as pulls wires) that can extend from a proximal end through the elongate shaft to the distal end of the elongate shaft. The elongate movement members can be manipulated, such as by manipulators on the one or more robotic arms 104, to control actuation of the elongate movement members.

In some implementations, the medical instrument 106 may include one or more sensors, such as electromagnetic (EM) sensors, shape sensors (such as shape sensing fiber), accelerometers, gyroscopes, satellite-based positioning sensors (such as global positioning system (GPS) sensors), or radio-frequency (RF) transceivers, among other examples. The sensors can be configured to generate or produce sensor data or provide the sensor data to another device or component. The sensors can be disposed at a distal end of the elongate shaft or along a length of the elongate shaft. In some implementations, the medical instrument 106 may be configured to receive an elongate member or device through a working channel, wherein the elongate member includes one or more sensors along a length of the elongate member. One or more sensors on the medical instrument 106 may provide sensor data to control circuitry of the medical system 100, which is then used to determine a position, orientation, or shape of the medical instrument 106.

The medical system 100 can also include a control system 118 (also referred to as a “control tower” or “mobile tower”). The control system 118 can be communicatively coupled (such as via wired or wireless connections) to the robotic system 102 to control various aspects of the robotic system 102 (such as electronics, optics, sensors, or power) or one or more subsystems associated with the robotic system 102, such as a fluid management system (not shown). Placing such functionality in the control system 118 can allow for a smaller form factor of the robotic system 102 that may be more easily adjusted or re-positioned by an operator or user. Additionally, the division of functionality between the robotic system 102 and the control system 118 can reduce operating room clutter and facilitate efficient clinical workflow.

The medical system 100 can include an electromagnetic (EM) field generator 120, which is configured to broadcast or emit an EM field that can be detected by various EM sensors, such as a sensor disposed on the medical instrument 106. The EM field can induce small electric currents in coils of the EM sensors, which can be analyzed to determine a position, angle, or orientation of the EM sensors relative to the EM field generator 120. Although EM fields and EM sensors are described in many examples herein, position sensing systems or sensors can include various other types of position sensing systems or sensors, such as optical position sensing systems or sensors, image-based position sensing systems or sensors, among other examples.

The medical system 100 can further include an imaging system 122 (also referred to as an “imaging device”) configured to generate, provide, or send image data (also referred to as “images”) to another device or system. For example, the imaging system 122 can generate image data depicting an anatomy of the patient 110 and provide the image data to the control system 118, the robotic system 102, or another device. The imaging system 122 may include an emitter or energy source (such as an X-ray source) or a detector (such as an X-ray detector) mounted on a C-shaped arm support 124, which allows for flexibility in positioning around the patient 110 to capture images from various angles without moving the patient 110. Use of the imaging system 122 can provide visualization of internal structures or anatomy, which can be used for a variety of purposes, including navigation of the medical instrument 106 (such as by providing images of internal anatomy to a user) and localization of the medical instrument 106 (based on an analysis of image data), among other examples. In some aspects, the imaging system 122 may enhance the efficacy or safety of a medical procedure, such as a bronchoscopy, by providing clear, continuous visual feedback to the operating surgeon or team.

In some implementations, the imaging system 122 may be a mobile device configured to move around an environment. For example, the imaging system 122 can be positioned next to the patient 110 (as shown in FIG. 1) during a particular phase of a procedure and removed when the imaging system 122 is no longer needed. In some other implementations, the imaging system 122 may be part of the table 112 or other equipment in an operating environment. The imaging system 122 can be implemented as a Computed Tomography (CT) machine or system, X-ray machine or system, fluoroscopy machine or system, Positron Emission Tomography (PET) machine or system, PET-CT machine or system, CT angiography machine or system, Cone-Beam CT (CBCT) machine or system, three-dimensional rotational angiography (3DRA) machine or system, single-photon emission computed tomography (SPECT) machine or system, Magnetic Resonance Imaging (MRI) machine or system, Optical Coherence Tomography (OCT) machine or system, or ultrasound machine or system, among other examples. In some implementations, the medical system 100 may include different types of imaging systems that can be used or positioned over the patient 110 during different phases or portions of a procedure depending on the needs at that time.

In some implementations, the imaging system 122 may be configured to process multiple images (also referred to as “image data”) to generate a three-dimensional (3D) view or model. For example, the imaging device 122 can be implemented as a CT machine configured to capture or generate a series of images (also referred to as “tomograms) or image data representing two-dimensional (2D) cross-sections or slices of a 3D volume from different angles around the patient 110, and then use one or more algorithms to reconstruct these images or image data into a 3D model. The 3D model can be provided to the control system 118, robotic system 102, or another device, such as for processing or display.

In some implementations, image data from the imaging system 122 may be used to localize various elements, such as the medical instrument 106, a target within the anatomy, or specific anatomical features, among other examples. As used herein, the terms “localize,” “localization,” or “localizing” refer to any processes for determining or estimating a position (or location) and/or orientation (or heading), collectively referred to as the “pose,” of the instrument or the target (or any other element) within a given space or environment. For example, the control system 118 can be configured to provide navigation information during a procedure to assist a user navigating the medical instrument 106 within the anatomy to reach a target (such as a desired treatment site or location). In some implementations, a target can include a nodule, such as in the context of certain bronchoscopy procedures. To illustrate, the control system 118 can display a navigation view or graphical data 126 that includes an instrument indicator 128 representing the medical instrument 106, a target indicator 130 representing the target, and an anatomical map. The navigation data 126(A) (such as initial navigation data) can be determined based on sensor data from a sensor of the medical instrument 106 (such as EM sensor data associated with the EM field generator 120), a map of the anatomy, or a location of the target. In some implementations, the map or location of the target may be determined based on preoperative data, such as data obtained during a preoperative procedure to find a target location or map the anatomy.

In some implementations, the navigation data 126(A) may be dynamically updated based on image data 132 from the imaging system 122. For example, the control system 118 can receive the image data 132 and analyze the image data 132 to determine a current or actual spatial relationship between the medical instrument 106 and the target. In some implementations, the control system 118 may display the image data 132 to a user, receive user input indicating a position of the medical instrument 106 or a position of the target in the image data 132, and analyze the image data 132 based on the user input to determine the current spatial relationship. If the control system 118 determines that the navigation data 126(A) incorrectly depicts the location of the medical instrument 106 (such as where the spatial relationship associated with the image data 132 is different than the spatial relationship associated with the navigation data 126(A)), the control system 118 may update the navigation data 126(A) at 134 and provide updated navigation data 126(B) that reflects the current or near real-time position of the medical instrument 106 relative to the target or the map.

The various components of the medical system 100 can be communicatively coupled to each other over a network, which can include a wireless or wired network. Example networks include one or more personal area networks (PANs), local area networks (LANs), wide area networks (WANs), Internet area networks (IANs), cellular networks, the Internet, personal area networks (PANs), body area network (BANs), etc. In some examples, various communication interfaces can include wireless technology, such as Bluetooth, Wi-Fi, near-field communication (NFC), or the like. Furthermore, in some examples, the various components of the medical system 100 can be connected for data communication, fluid exchange, power exchange, and so on, via one or more support cables, tubes, connections, or the like.

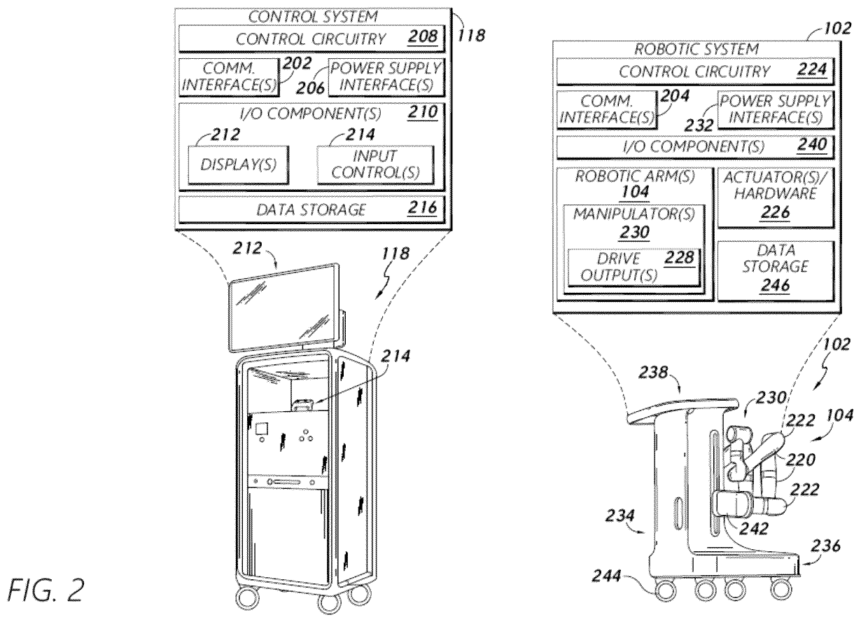

FIG. 2 shows example components of the control system 118 and the robotic system 102 of FIG. 1, according to some implementations. In the examples of FIG. 2, the control system 118 and the robotic system 102 are implemented as a tower and a robotic cart, respectively. However, the control system 118 and robotic system 102 can be implemented in other manners. The control system 118 can be coupled to the robotic system 102 and operate in cooperation therewith to perform a medical procedure. For example, the control system 118 can include communication interface(s) 202 for communicating with communication interface(s) 204 of the robotic system 102 via a wireless or wired connection (such as to control the robotic system 102). In some implementations, the control system 118 may communicate with the robotic system 102 to receive position or sensor data therefrom relating to the position of sensors associated with an instrument or member controlled by the robotic system 102. For example, the control system 118 may communicate with the EM field generator 120 to control generation of an EM field in an area around a patient. The control system 118 can further include one or more power supply interface(s) 206.

The control system 118 can include control circuitry 208 configured to cause one or more components of the medical system 100 to actuate or otherwise control any of the various system components, such as carriages, mounts, arms or positioners, medical instruments, imaging devices, position sensing devices, or sensors, among other examples. Further, the control circuitry 208 can be configured to perform other functions, such as cause display of information, process data, receive input, communicate with other components or devices, or any other function or operation described herein.

The control system 118 can further include one or more input or out (I/O) components 210 configured to assist a physician or others in performing a medical procedure. For example, the one or more I/O components 210 can be configured to receive input or provide output to enable a user to control or navigate the medical instrument 106, the robotic system 102, or other instruments or devices associated with the medical system 100. The control system 118 can include one or more displays 212 to provide, display or otherwise present various information regarding a procedure. For example, the one or more displays 212 can be used to present navigation information including a virtual anatomical model of anatomy with a virtual representation of a medical instrument, image data, or other information. The one or more I/O components 210 can include one or more user input control(s) 214, which can include any type of user input (or output) devices or device interfaces, such as one or more buttons, keys, joysticks, handheld controllers (such as video-game-type controllers), computer mice, trackpads, trackballs, control pads, sensors (such as motion sensors or cameras) that capture hand gestures and finger gestures, touchscreens, toggle (such as button) inputs, or interfaces or connectors therefore. In some implementations, such inputs can be used to generate commands for controlling one or more medical instruments, robotic arms, or other components.

The control system 118 can also include data storage 216 configured to store executable instruments (such as computer-readable instructions) that can be executed by the control circuitry 208 to cause the control circuitry 208 to perform various operations or functionality described herein. In some implementations, the data storage 216 also may store telemetry or runtime data (such as sensor data or image data) generated by the medical system 100 or otherwise captured or acquired during a medical procedure. In some implementations, two or more components of the control system 118 can be electrically or communicatively coupled to each other.

The robotic system 102 can include the one or more robotic arms 104 configured to engage with or control, for example, the medical instrument 106 or other elements or components to perform one or more aspects of a procedure. As shown in FIG. 2, each robotic arm 104 can include multiple segments 220 coupled to joints 222, which can provide multiple degrees of movement or freedom. The robotic system 102 can be configured to receive control signals from the control system 118 to perform certain operations, such as to position one or more of the robotic arms 104 in a particular manner or manipulate an instrument, among other examples. In response, the robotic system 102 can control, using control circuitry 224 thereof, actuators 226 or other components of the robotic system 102 to perform the operations. For example, the control circuitry 224 can control insertion or retraction, articulation, or roll of a shaft of the medical instrument 106 or other instrument by actuating one or more drive outputs 228 of a manipulator 230 (or end-effector) coupled to a base of a robotically-controllable instrument. The drive outputs 228 can be coupled to a drive input on an associated instrument, such as an instrument base of an instrument that is coupled to the associated robotic arm 104. The robotic system 102 also may include one or more power supply interfaces 232.

The robotic system 102 can include a support column 234, a base 236, or a console 238. The console 238 can provide one or more I/O components 240, such as a user interface for receiving user input or a display screen (or a dual-purpose device, such as a touchscreen) to provide the physician or user with preoperative or intraoperative data. The support column 234 can include an arm support 242 (also referred to as a “carriage”) for supporting the deployment of the one or more robotic arms 104. The arm support 242 can be configured to vertically translate along the support column 234. Vertical translation of the arm support 242 allows the robotic system 102 to adjust the reach of the robotic arms 104 to meet a variety of table heights, patient sizes, or physician preferences. The base 236 can include wheel-shaped casters 244 (also referred to as “wheels”) that allow the robotic system 102 to move around the operating room. After reaching the appropriate position, the casters 244 can be immobilized using wheel locks to hold the robotic system 102 in place during the procedure.

The joints 222 of each robotic arm 104 can each be independently-controllable or provide an independent degree of freedom available for instrument navigation. In some implementations, each robotic arm 104 may include seven joints that provide seven degrees of freedom, including “redundant” degrees of freedom. Redundant degrees of freedom can allow robotic arms 104 to be controlled to position their respective manipulators 230 at a specific position, orientation, or trajectory in space using different linkage positions and joint angles. This allows for the robotic system 102 to position or direct a medical instrument from a desired point in space while allowing the physician to move the joints 222 into a clinically advantageous position away from the patient to create greater access, while avoiding collisions.

The one or more manipulators 230 (or end-effectors) can be coupled to an instrument base or handle, which can be attached using a sterile adapter component. The combination of the manipulator 230 and instrument base, as well as any intervening mechanics or couplings (such as the sterile adapter), can be collectively referred to as the manipulator or a manipulator assembly. Manipulators or manipulator assemblies can provide power or control interfaces. Example interfaces may include connectors to transfer pneumatic pressure, electrical power, electrical signals, or optical signals from the robotic arm 104 to an instrument base. Manipulators or manipulator assemblies can be configured to manipulate medical instruments (such as surgical tools) using techniques including, for example, direct drives, harmonic drives, geared drives, belts or pulleys, or magnetic drives, among other examples.

The robotic system 102 can also include data storage 246 configured to store executable instruments (such as computer-readable instructions) that can be executed by the control circuitry 224 to cause the control circuitry 224 to perform various operations or functionality described herein. In some implementations, the data storage 216 also may store telemetry or runtime data (such as sensor data or image data) generated by the medical system 100 or otherwise captured or acquired during a medical procedure. In some implementations, two or more of the components of the robotic system 102 can be electrically or communicatively coupled to each other.

Data storage (including the data storage 216, data storage 246, or other data storage or memory) can include any suitable or desirable type of computer-readable media. For example, computer-readable media can include one or more volatile data storage devices, non-volatile data storage devices, removable data storage devices, or nonremovable data storage devices implemented using any technology, layout, or data structure(s) or protocol, including any suitable or desirable computer-readable instructions, data structures, program modules, or other types of data.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Functionality described herein can be implemented by the control circuitry 208 of the control system 118 or the control circuitry 224 of the robotic system 102, such as by the control circuitry 208 or 224 executing instructions to cause the control circuitry 208 or 224 to perform the functionality. Control circuitry (including the control circuitry 208, control circuitry 224, or other control circuitry) can include circuitry embodied in a robotic system, control system or tower, instrument, or any other component or device. Control circuitry can include any collection of processors, processing circuitry, processing modules or units, chips, dies (such as semiconductor dies including one or more active or passive devices or connectivity circuitry), microprocessors, micro-controllers, digital signal processors, microcomputers, central processing units, field-programmable gate arrays, programmable logic devices, state machines (such as hardware state machines), logic circuitry, analog circuitry, digital circuitry, or any device that manipulates signals (analog or digital) based on hard coding of the circuitry or operational instructions.

Control circuitry referenced herein can further include one or more circuit substrates (such as printed circuit boards), conductive traces and vias, or mounting pads, connectors, or components. Control circuitry can further include one or more storage devices, which may be embodied in a single device, a plurality of devices, or embedded circuitry of a device. Such data storage can comprise read-only memory, random access memory, volatile memory, non-volatile memory, static memory, dynamic memory, flash memory, cache memory, data storage registers, or any device that stores digital information. In examples in which control circuitry includes a hardware or software state machine, analog circuitry, digital circuitry, or logic circuitry, data storage device(s) or register(s) storing any associated operational instructions can be embedded within, or external to, the circuitry comprising the state machine, analog circuitry, digital circuitry, or logic circuitry.

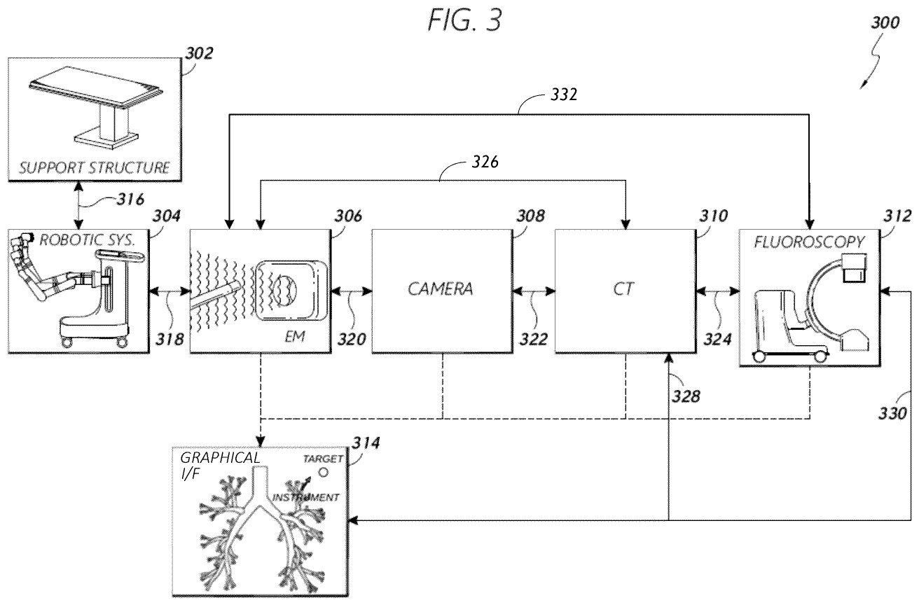

FIG. 3 shows a block diagram of an example localization system 300, according to some implementations. The localization system 300 includes various positioning or imaging systems or modalities 302-312 (also referred to as “subsystems”), which can be implemented to facilitate anatomical mapping, navigation, positioning, or visualization for procedures in accordance with one or more examples. For example, the various systems 302-312 can be configured to provide data for generating an anatomical map, determining a location of an instrument, determining a location of a target, or performing other techniques.

Each of the systems 302-312 can be associated with a respective coordinate space (also referred to as a “position coordinate frame”) or can provide data or information relating to instrument or anatomy locations, wherein registering the various coordinate spaces to one another can allow for integration of the various systems to provide mapping, navigation, or instrument visualization. For example, registering a first modality to a second modality can allow for determined positions in the first modality to be tracked or superimposed on or in a reference frame associated with the second modality, thereby providing layers of positional information that can be combined to provide a robust localization system.

In some aspects, the system 300 may be configured to perform one or more localization or localizing techniques. In some implementations, the anatomical space in which a medical instrument can be localized (such as where a pose or shape of the instrument is determined or estimated) may be a 2D or 3D portion of a patient's tracheobronchial airways, vasculature, urinary tract, gastrointestinal tract, or any organ or space accessed via lumens. Various modalities can be implemented to provide images, representations, or models of the anatomical space. For example, an imaging modality can be implemented, which can include, for example, X-ray, fluoroscopy, CT, PET, PET-CT, CT angiography, CBCT, 3DRA, SPECT, MRI, OCT, or ultrasound, among other examples. In some implementations, the imaging modality may be used to capture or acquire images of a patient's anatomy during a preoperative phase of a medical procedure. In some other implementations, the imaging modality may be used to capture or acquire images of a patient's anatomy during an intraoperative phase of the medical procedure.

The systems 302-312 can provide information for generating a graphical user interface 314 (also referred to as a “graphical interface (I/F)”) that includes navigation information for navigating an instrument to a target within an anatomy (such as the navigation data 126(A) or 126(B) of FIG. 1). For example, the navigation information may include an anatomical map, an estimated position, orientation, and/or shape of the instrument. The navigation information also may include a shape, boundary, eccentricity, texture, and/or position of the target. In some implementations, the graphical user interface 314 or other localization information may be displayed to a user, such as a physician, during a medical procedure to assist the user in performing the procedure. For example, a visualization of a tracked instrument can be superimposed on an anatomical map depicted by the graphical user interface 314 based on position or sensor data associated with the tracked medical instrument.

As shown in FIG. 3, the system 300 can include a support structure 302 (such as a surgical bed or other patient positioning or support platform). For example, the support structure 302 includes a planar surface that contacts and supports the patient. In some implementations, the position of the support structure 302 may be known based on data maintained relating to the position of the support structure 302 within the surgical or procedure environment. In some other implementations, the position of the support structure 302 may be sensed or otherwise determined using one or more markers or an appropriate imaging or positioning modality.

The system 300 can further include a robotic system 304 (such as a robotic cart or other device or system including one or more robotic end effectors). In some implementations, the robotic system 304 may be one example of the robotic system 102 of FIGS. 1 and 2. Data relating to the position or state of robotic arms, actuators, or other components of the robotic system 304 can be known or derived from robotic command data or other robotic data relative to a coordinate frame of the robotic system 304. In some examples, reference frame registration 316 occurs between the support structure 302 and the robotic system 304, which can be a relatively coarse registration, in some implementations, based on robotic system or cart-set-up procedure (which can have any suitable or desirable scheme).

The system 300 can further include an electromagnetic (EM) sensor system 306, which can include an EM field generator (such as the EM field generator 120 of FIG. 1) and one or more EM sensors. An EM sensor can be associated with a portion of an instrument that is tracked or controlled, such as a distal end (or tip) of the instrument or along a length of the instrument or other elongate member (such as a working channel) disposed in a lumen of the instrument. In some implementations, the EM field generator can be mechanically coupled to the support structure 302 or the robotic system 304 such that registration or association 318 between such systems can be known or determined. In some implementations, the registration 318 between the EM sensor system 306 and the robotic system 304 can be determined through forward kinematics or field generator mount transform information. For example, the field generator can be mounted to the support structure 302 such that the position of the field generator can be known relative to the robotic system positioning frame based on a known relationship between the position of the support structure 302 and the robotic system 304. The EM sensor system 306 can provide instrument pose or path information based on sensor readings associated with the instrument.

The system 300 can further include an optical camera system 308 including one or more cameras or other imaging devices configured to generate images of patient anatomy within a visual field thereof (such as real-time image data) during a surgical procedure. In some implementations, registration 320 between the optical camera system 308 and the EM sensor system 306 can be achieved through identification of features having EM sensor data associated therewith, such as a medical instrument tip, in images generated by the optical camera system 308. The registration 320 can further be based at least in part on hand-eye interaction of the physician when viewing real-time camera images while the EM-sensor-equipped endoscope is navigating in the patient anatomy.

The system 300 can further include a computed tomography (CT) imaging system 310 configured to generate CT images of the patient anatomy, which can be performed preoperatively or intraoperatively. The CT imaging system 310 is generally used for scanning a relatively large volume. In some implementations, image processing can be implemented for registration 322 of the CT image data with the camera image data generated by the optical camera system 308. For example, common features identified in both camera image data and CT image data can be identified to relate the CT image frame to the camera image frame in space. In some examples, the CT imaging system 310 can be used to generate preoperative imaging data for producing the graphical user interface 314 or for path navigation planning.

In some aspects, the CT imaging system 310 may be registered 326 to the EM sensor system 306 through various techniques. In some implementations, a mechanical structure of the CT imaging system 310 can have a known physical transform or relationship with respect to a mounting position of the EM field generator of the EM sensor system 306. Such known relationship can be used to register the CT image space to the EM sensor space. The connection 328 represents a mapping or relationship between the CT imaging system 310 and an anatomical map depicted by a graphical user interface 314.

The system 300 can further include a fluoroscopy imaging system 312 configured to generate tomographic images (such as real-time X-ray images) of the surgical site. The fluoroscopy imaging system 312 is generally used for scanning a smaller volume compared to the CT imaging system 310. In some implementations, the fluoroscopy imaging system 312 may be one example of the imaging system 122 of FIG. 1. For example, the fluoroscopy imaging system 312 may include a CBCT scanner coupled to a C-arm. In some implementations, the fluoroscopy imaging system 312 may be used with a contrast agent introduced into the anatomy to generate image data representing patient anatomy or instrumentation. In some implementations, the fluoroscopy imaging system 312 may be registered 324 to the CT imaging system 310 using any image processing technique suitable for such registration.

In some aspects, the fluoroscopy imaging system 312 may be registered 332 to the EM sensor system 306 through various techniques. In some implementations, a mechanical structure of the fluoroscopy imaging system 312 (such as the C-arm instrumentation) can have a known physical transform or relationship with respect to a mounting position of the EM field generator of the EM sensor system 306. Such known relationship can be used to register the fluoroscopy image space to the EM sensor space. The connection 330 represents a mapping or relationship between the fluoroscopy imaging system 312 and an anatomical map depicted by the graphical user interface 314.

In the example of FIG. 3, the CT imaging system 310 and fluoroscopy imaging system 312 are illustrated as separated systems. However, in some other implementations, a single imaging system may perform the functions of both the CT imaging system 310 and fluoroscopy imaging system 312.

The position, shape, or orientation of an instrument, such as an endoscope, can be determined using any one or more of the systems 302-312, which can facilitate generation of graphical interface data representing the estimated position or shape of the instrument relative to an anatomical map depicted by the graphical user interface 314. The graphical user interface 314 can be displayed on a display device, such as via the control system 118 or robotic system 102, or another device. In some implementations, the graphical user interface 314 also may indicate a position of a target within the anatomy that has been designated for treatment.

Although the systems 302-312 have been described in a particular order, the operations or functions associated therewith can be performed in different orders. In some implementations, the systems 302-312 can be used in different ways. In some other implementations, registration can occur between different systems and modalities.

In some aspects, one or more of the systems 302-312 may be used to generate the graphical user interface 314 preoperatively or determine a location of one or more targets within an anatomical map depicted by the graphical user interface 314 during a preoperative phase of a medical procedure. As used herein, the terms “skeleton image data” and “skeleton image space” refer to images of an anatomy captured or acquired by an imaging system (such as the CT imaging system 310) during the preoperative phase of a medical procedure. However, a graphical user interface 314 generated using a preoperative CT scan may not accurately reflect the spatial relationship between a medical instrument and a target during an intraoperative phase. For example, changes in the patient's anatomy or the medical environment can cause the spatial relationship between the instrument and the target to deviate from what is depicted in the graphical user interface 314. Example factors that may cause such deviations include EM distortion, poor registration (or mapping) between the sensor data and the image data, outdated preoperative scans, and anatomical deformations, among other examples. Thus, in some other aspects, one or more of the systems 302-312 may be used to determine a location of a medical instrument or position of a target relative to an anatomical map depicted by the graphical user interface 314 during an intraoperative phase of the medical procedure.

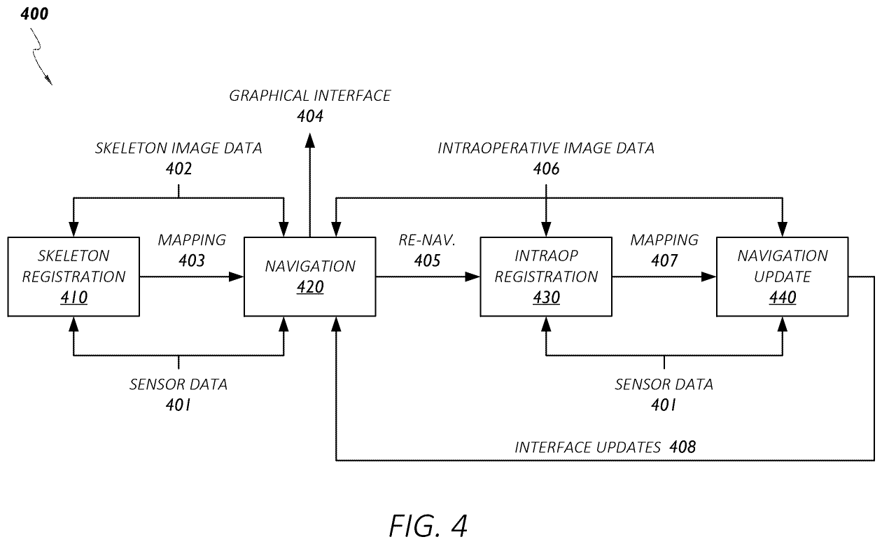

FIG. 4 shows a block diagram of an example navigation system 400, according to some implementations. In some aspects, the navigation system 400 may be implemented by a medical system such as the medical system 100 of FIG. 1. With reference to FIG. 2, the navigation system 400 may be one example of any of the control circuitry 208 or 224. The navigation system 400 is configured to generate or update a graphical interface 404 that depicts or otherwise indicates a spatial relationship between a medical instrument and a target within an anatomy. For example, the graphical interface 404 may be used by the medical system or a user thereof (such as a physician) to navigate the instrument to the target. In some implementations, the graphical interface 404 may be one example of the graphical user interface 314 of FIG. 3.

In some aspects, the navigation system may generate the graphical interface 404 based at least in part on image data 402 (also referred to as “skeleton image data”) acquired via an imaging system (such as the CT imaging system 310 of FIG. 3) and sensor data 401 acquired via a sensor system (such as the EM sensor system 306 of FIG. 3). For example, the skeleton image data 401 may include one or more tomograms captured as a result of a CT scan (such as described with reference to FIGS. 1-3). In some implementations, the skeleton image data 401 may be captured during a preoperative phase of a medical procedure. The sensor data 401 may indicate a pose of the instrument and/or one or more EM sensors disposed in an EM field (such as described with reference to FIGS. 1-3). As used herein, the term “pose” may refer to a position and/or orientation of a sensor or an instrument. In some implementations, the pose of the instrument may be determined based at least in part on sensor data 401 received from a sensor disposed on the instrument (such as at the distal end of an endoscope).

The navigation system 400 includes a skeleton registration component 410, a navigation component 420, an intraoperative registration component 430, and a navigation update component 440. The skeleton registration component 410 is configured to register a coordinate space associated with the skeleton image data 402 (also referred to as the “skeleton image space”) to a coordinate space associated with the sensor data 401 (also referred to as the “sensor space”). As used herein, the term “registration” refers to a mapping between different coordinate spaces. In other words, the skeleton registration component 410 may determine a mapping 403, based at least in part on the sensor data 401 and the skeleton image data 402, that can transform any data point in the sensor space to a respective data point in the image space (such as a transformation matrix).

Various existing techniques can be used to register the skeleton image space with the sensor space, and vice-versa. For example, a number of EM sensors (such as 3 or more EM sensors) may be placed around a patient's body at predetermined positions relative to the anatomy. The skeleton registration component 410 may determine the positions of the EM sensors in an EM field (which represents the sensor space) based on the received sensor data 401. The skeleton registration component 410 also may determine corresponding positions for the EM sensors in the skeleton image space based on their relative locations to known anatomical features depicted by the skeleton image data 402. The skeleton registration component 410 may further determine a mapping 403 (or transformation matrix) that transforms the positions of the EM sensors in the sensor space to their respective positions in the skeleton image space.

The navigation component 420 is configured to generate the graphical interface 404 based on the sensor data 401, the skeleton image data 402, and the mapping 403 between the sensor space and the skeleton image space. The graphical interface 404 may indicate a spatial relationship between the instrument and the target within the anatomy. For example, the graphical interface 404 may include a three-dimensional (3D) model of the anatomy for display on one or more displays associated with the medical system (such as the displays 212 of FIG. 2). In some implementations, the navigation component 420 may render the 3D model based at least in part on the skeleton image data 402. Accordingly, the 3D model of the anatomy may provide a visual indicator of the location of the target within the anatomy. In some aspects, the graphical interface 404 may be used by the medical system or a user (such as a physician) to navigate the instrument to the target during the intraoperative phase of the medical procedure.

In some aspects, the navigation component 420 also may display a pose and/or path of the instrument on the 3D model of the anatomy. For example, the navigation component 420 may determine the pose of the instrument in relation to the anatomy based on the sensor data 401 and the mapping 403 between the sensor space and the skeleton image space. More specifically, the navigation component 420 may use the mapping 403 to transform the pose of the instrument in the sensor space (as indicated by the sensor data 401) to a corresponding pose in the skeleton image space. The navigation component 420 may continuously track any changes in the pose or movement of the instrument based on the sensor data 401 and update the graphical interface 404 to reflect the real-time pose of the instrument in relation to the anatomy.

In some aspects, the navigation component 420 may analyze a spatial relationship between the instrument and the target to provide guidance for navigating the instrument within the anatomy. For example, the navigation component 420 may plan a navigation path through the patient's anatomy from the instrument to the target. The navigation path may be rendered or displayed on the graphical interface 404 for the instrument to follow. In some implementations, the navigation component 420 may determine the position of the target in the skeleton image space based at least in part on the skeleton image data 402. In some other implementations, the navigation component 420 may determine the position of the target in the skeleton image space based at least in part on input provided by a user of the navigation system 400. For example, the user may specify or otherwise input the location of the target on the 3D model of the anatomy.

As described above, a graphical user interface 404 generated using image data acquired during a preoperative phase of a medical procedure may not accurately reflect the spatial relationship between a medical instrument and a target during an intraoperative phase. For example, changes in the patient's anatomy or the medical environment can cause the spatial relationship between the instrument and target to deviate from what is depicted by the graphical interface 404 at any given time. Example factors that may cause such deviations include EM distortion, poor registration (or mapping 403) between the sensor space and the skeleton image space, outdated skeleton image data 402, and anatomical deformations, among other examples. In some aspects, the navigation component 420 may acquire updated images of the patient's anatomy captured via an intraoperative imaging system (such as the fluoroscopy imaging system 312 of FIG. 3) and use the updated image data 406 (also referred to as “intraoperative image data”) to improve or update the depiction of the spatial relationship between the instrument and the target in the graphical interface 404.

In some aspects, the navigation component 420 may be configured to operate in a “navigation mode” or a “re-navigation mode” depending on whether a correction is applied to the graphical interface 404 based on the intraoperative image data 406. For example, the navigation component 420 may operate in the navigation mode while the instrument is initially navigated to a threshold proximity of the target prior to acquiring the intraoperative image data 406. When navigating the instrument past the threshold, the navigation component 420 may be configured to operate in the re-navigation mode. In some aspects, the operating mode of the navigation component 420 may be manually toggled by a user of the navigation system 400. For example, the user may dynamically switch between the navigation mode and the re-navigation mode at any given time during a medical procedure (provided certain preconditions are met). In some implementations, the navigation component 420 may pause 405 navigation or robot motion so that the intraoperative imaging system can be brought to a location proximate the anatomy to capture or acquire the intraoperative image data 406.

The intraoperative registration component 430 is configured to register a coordinate space associated with the intraoperative image data 406 (also referred to as the “intraoperative image space”) with the sensor space responsive to the navigation component 420 signaling a pause 405 in navigation. More specifically, the intraoperative registration component 430 may determine a mapping 407, based at least in part on the sensor data 401 and the intraoperative image data 406, that can transform any data point in the intraoperative image space to a respective data point in the sensor space. In some aspects, the intraoperative registration component 430 may determine the mapping 407 based at least in part on a pose of the instrument in the sensor space and a corresponding instrument pose in the intraoperative image space.

In some implementations, the intraoperative registration component 430 may determine the pose of the instrument in the intraoperative image space through analysis of the intraoperative image data 406 using one or more image processing techniques. Example suitable image processing techniques include segmentation, machine learning, and statistical analysis, among other examples. As used herein, the term “segmentation” refers to various techniques for partitioning a digital image into groups of voxels (or “image segments”) based on related characteristics or identifying features. In some implementations, the intraoperative registration component 430 may segment the intraoperative image data 406 so that the pose of the instrument can be detected or estimated from the corresponding images (or tomograms).

The intraoperative registration component 430 may determine the pose of the instrument in the sensor space based at least in part on the sensor data 401. Aspects of the present disclosure recognize that some imaging technologies (such as CT or X-rays) may interfere with some sensor technologies (such as EM). Thus, in some implementations, the intraoperative registration component 430 may determine the pose of the instrument in the sensor space based on sensor data 401 captured or acquired while the intraoperative imaging system cannot interfere with the underlying sensor technology (such as while the fluoroscopy imaging system 312 is located outside the EM field of the EM sensor system 306).

The navigation update component 440 is configured to determine one or more interface updates 408 to be applied to the graphical interface 404 based on the intraoperative image data 406 and the mapping 407. In some implementations, the interface updates 408 may include a position of the target transformed from the intraoperative image space to the sensor space. In some other implementations, the interface updates 408 may include a pose of the instrument transformed from the intraoperative image space to the sensor space. For example, the navigation update component 440 may determine the target position or the instrument pose in the intraoperative image space through segmentation of the intraoperative image data 406 (such as described above). The navigation update component 440 may further use the mapping 407 to transform the target position or instrument pose in the intraoperative image space to a corrected target position or instrument pose, respectively, in the sensor space. As used herein, the terms “corrected target position” and “corrected instrument pose” refer to the target position and instrument pose that are transformed from the intraoperative image space to the sensor space. The corrected target position and/or the corrected instrument pose in the sensor space may be provided, as the interface updates 408, to the navigation component 420.

In some implementations, the navigation component 420 may update the graphical interface 404 based at least in part on the interface updates 408. More specifically, the navigation component 420 may update the spatial relationship between the instrument and the target depicted by the graphical interface 404. In some aspects, the navigation component 420 may update the spatial relationship in the sensor space. For example, the navigation component 420 may use the mapping 403 to transform the position of the target from the skeleton image space to the sensor space. Assuming the physical position of the target remains unchanged from the preoperative phase of the medical procedure to the intraoperative phase, the navigation component 420 may determine a transformation (T^{corr}) that maps the position of the target in the sensor space to the corrected target position indicated by the interface updates 408. The navigation component 420 may apply the same transformation T^{corr} to the sensor data 401 to correct the spatial relationship between the instrument and the target.

In some other aspects, the navigation component 420 may update the spatial relationship between the instrument and the target in the skeleton space. For example, the navigation component 420 may determine a correction to be applied to the position of the target in the skeleton image space based on the interface updates 408. In some implementations, the navigation component 420 may use the mapping 403 to transform the corrected target position in the sensor space to a corresponding position in the skeleton image space and determine a transformation (tₙ_{^{SKEL}}^{corr}) between the current position of the target in the skeleton image space and the corrected target position. The navigation component 420 may further apply a correction transformation (T_{S}_{^{SKEL}}^{corr}), based on the transformation tₙ_{^{SKEL}}^{corr}, to the sensor data 401 to correct the pose of the instrument relative to the position of the target in the graphical interface 404.

By updating the spatial relationship between the instrument and the target based on the intraoperative image data 406 acquired during the intraoperative phase, the navigation system 400 can provide more accurate navigation information to the medical system or user. In some aspects, the navigation system 400 may dynamically update the graphical interface 404 based on new intraoperative image data 406. For example, the intraoperative imaging system may be used to scan the anatomy multiple times throughout the intraoperative phase. Each time the anatomy is scanned, the intraoperative registration component 430 may determine a new mapping 407 based on the most recent intraoperative image data 406 and the sensor data 401, and the navigation update component 440 may use the updated mapping 407 to generate new interface updates 408.

As described with reference to FIG. 4, the navigation component 420 may dynamically switch between the navigation mode and the re-navigation mode at any given time. For example, if the user is unsatisfied with the spatial relationship depicted by the graphical interface 404 in the re-navigation mode, the user may toggle the navigation component 420 to revert back to the standard navigation mode at any time. In some implementations, the navigation component 420 may display a user interface feature on the graphical interface 404 that allows a user to toggle between the navigation mode and the re-navigation mode. More specifically, the user interface feature may allow the user to dynamically apply or remove a correction to real-time sensor data 401 based on the interface updates 408 without reinitializing navigation or otherwise disrupting the workflow during the intraoperative phase. For example, reinitializing navigation may require backing the instrument out of the anatomy, recalibrating the mapping 403 between the sensor space and the skeleton image space, and driving the instrument to the target once again.

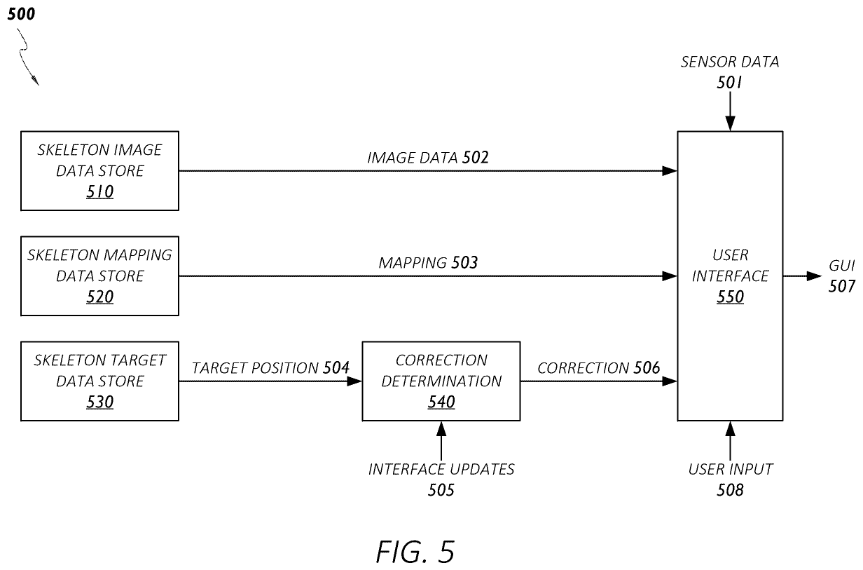

FIG. 5 shows another block diagram of an example navigation system 500, according to some implementations. The navigation system 500 is configured to generate a graphical user interface (GUI) 507 that depicts or otherwise indicates a spatial relationship between a medical instrument and a target within an anatomy based on real-time sensor data 501. In some implementations, the navigation system 500 may be one example of the navigation system 400 of FIG. 4. With reference to FIG. 4, the GUI 507 may be one example of the graphical interface 404 and the sensor data 501 may be one example of the sensor data 401.

The navigation system 500 includes a skeleton image data store 510, a skeleton mapping data store 520, a skeleton target data store 530, a correction determination component 540, and a user interface component 550. The skeleton image data store 510 is configured to store image data 502 captured or acquired via an imaging system during a preoperative phase of a medical procedure (such as the CT imaging system 310 of FIG. 3). The skeleton mapping data store 520 is configured to store a mapping 503 that transforms any data point in a coordinate space associated with the sensor data 501 (also referred to as the “sensor space”) to a respective data point in a coordinate space associated with the image data 502 (also referred to as the “skeleton image space”), or vice-versa. In some implementations, the image data 502 and the mapping 503 may be examples of the skeleton image data 402 and the mapping 403, respectively, of FIG. 4.

The skeleton target data store 530 is configured to store a position 504 of a target in the skeleton image space. In some implementations, the navigation component 500 may determine the target position 504 based at least in part on the image data 502. For example, the navigation component 500 may analyze the image data 502 using one or more image processing techniques to determine the target position 504. Example suitable image processing techniques include segmentation, machine learning, and statistical analysis, among other examples. In some other implementations, the navigation component 500 may determine target position 504 based at least in part on input provided by a user of the navigation system 500. For example, the user may specify or otherwise input the location of the target on a 3D model of the anatomy.

The correction determination component 540 is configured to determine a correction 506 to be applied to the sensor data 501 based at least in part on the target position 504 and one or more interface updates 505. In some implementations, the interface updates 505 may be one example of the interface updates 408 of FIG. 4. For example, the interface updates 505 may include a position of the target (or instrument) transformed from a coordinate space associated with image data acquired by an imaging system during an intraoperative phase of the medical procedure (such as the fluoroscopy system 312 of FIG. 3) to the sensor space. As described with reference to FIG. 4, the correction determination component 540 may determine or calculate the correction 506 based on a difference or offset between the target position 504 and the “corrected” target position indicated by the interface updates 505.

In some aspects, the correction determination component 540 may receive new interface updates 505 (such as from the navigation update component 440 of FIG. 4) in response to subsequent scans of the anatomy during the intraoperative phase (such as described with reference to FIG. 4). In some implementations, the correction determination component 540 may store or use only the most recent interface updates 505 in determining the correction 506 to be applied to the sensor data 501. In some other implementations, the correction determination component 540 may store a record of multiple interface updates 505. In such implementations, the correction determination component 540 may select one of the stored interface updates 505 to be used in determining the correction 506, for example, based on user input 508.

The user interface component 550 is configured to generate the GUI 507 based at least in part on the sensor data 501, the image data 502, the mapping 503, and the correction 506. In some aspects, the GUI 507 may include a navigation feature that depicts a spatial relationship between the instrument and the target. For example, the user interface component 550 may generate a 3D model of the anatomy based on the image data 502 and use the mapping 503 to superimpose a rendering or other visual depiction of the instrument onto the model based at least in part on an instrument pose indicated by the sensor data 501. The user interface component 550 is also configured to receive user input 508 via one or more input devices (not shown for simplicity). Example suitable input devices may include touchscreens, touchpads, buttons, switches, mice, keyboards, keypads, joysticks, and scroll wheels, among other examples.

In some aspects, the user interface component 550 may selectively apply the correction 506 to the sensor data 501 based on the user input 508. For example, the user interface component 550 may display a toggle feature on the GUI 507 that allows a user of the navigation system 500 to toggle the application of the correction 506 at any time (provided a correction 506 is available). If the user input 508 indicates a desire to correct the sensor data 501, the user interface component 550 may apply the correction 506 to the sensor data 501 and update the navigation feature in the GUI 507 to reflect an updated spatial relationship between the instrument and the target based on the corrected sensor data. On the other hand, if the user input 508 indicates a desire to un-correct the sensor data 501 (or to leave the sensor data 501 uncorrected), the user interface component 550 may refrain from applying the correction 506 to the sensor data 501 in determining the instrument pose to be depicted in the navigation feature.

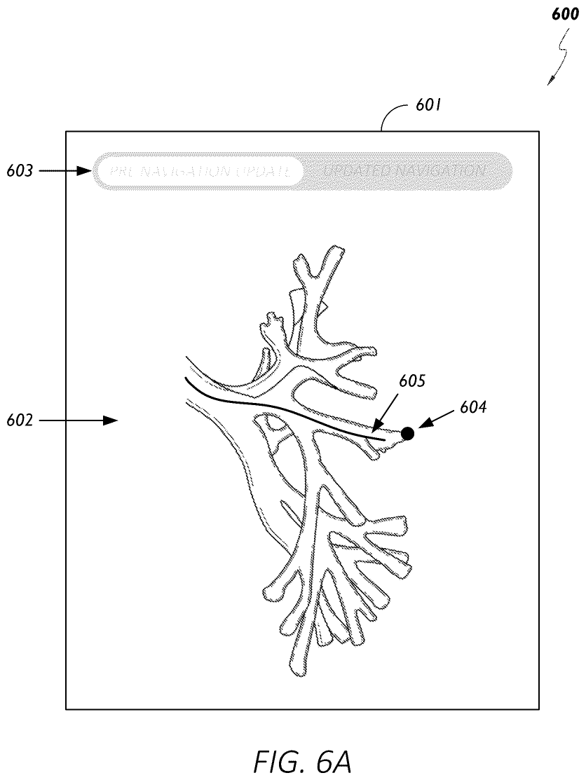

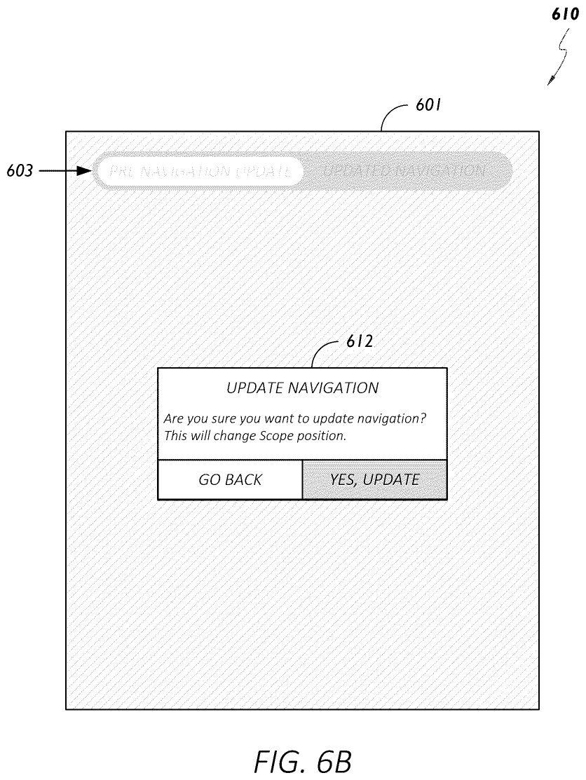

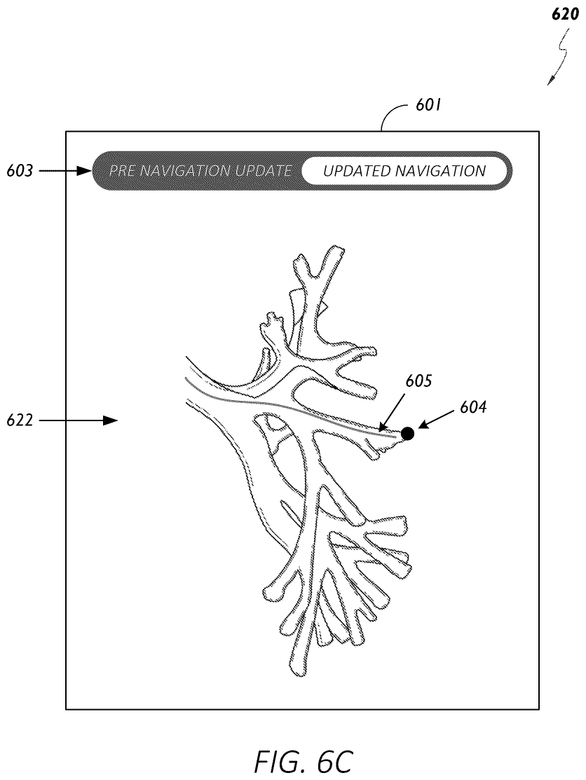

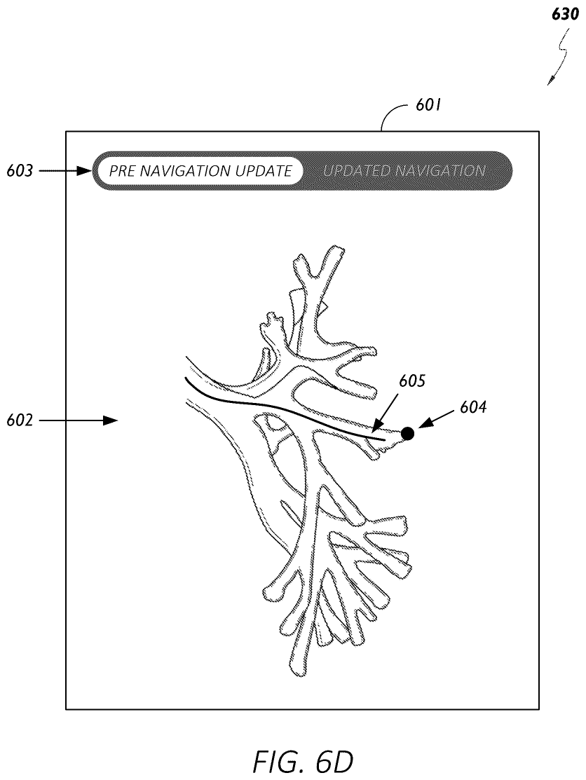

FIGS. 6A-6D show various states 600-630, respectively, of an example GUI 601 for navigating an instrument to a target within an anatomy, according to some implementations. In some implementations, the GUI 601 may be one example of the GUI 507 of FIG. 5. More specifically, FIG. 6A shows an example state 600 of the GUI 601 prior to registering the intraoperative image space with the sensor space (also referred to herein as “intraoperative registration”), FIG. 6B shows an example state 610 of the GUI 601 immediately preceding a procedure for registering the intraoperative image space with the sensor space, FIG. 6C shows an example state 620 of the GUI 601 following intraoperative registration, and FIG. 6D shows another example state 630 of the GUI 601 following intraoperative registration.

With reference to FIG. 6A, the GUI 601 is shown to include a navigation feature 602 depicting a spatial relationship between an instrument 605 and a target 604 within an anatomy. In the example of FIGS. 6A-6B, the instrument 605 is depicted as an endoscope and the target 604 is depicted as a nodule in a patient's lung (such as during a bronchoscopy). However, in actual implementations, the navigation feature 602 may depict any suitable instrument and/or target associated with various other types of medical procedures. The GUI 601 also includes a toggle feature 603 for toggling between a PRE NAVIGATION UPDATE view and an UPDATED NAVIGATION view.

In the example of FIGS. 6A-6D, the PRE NAVIGATION UPDATE view may cause the GUI 601 to display the navigation feature 602 showing a spatial relationship between the instrument 605 and the target 604 based on uncorrected sensor data. By contrast, the UPDATED NAVIGATION view may cause the GUI 601 to display an updated navigation feature 622 showing a spatial relationship between the instrument 605 and the target 604 based on corrected sensor data. Because FIG. 6A depicts the state 600 of the GUI 601 prior to intraoperative registration, the toggle feature 603 is currently disabled or otherwise locked to the PRE NAVIGATION UPDATE view.

With reference to FIG. 6B, the GUI 601 is shown to display a prompt 612 to a user indicating that a navigation update is now available. In some implementations, the prompt 612 may present the user with an option to proceed with updating the navigation, such as by clicking or tapping on a YES, UPDATE button in the bottom right corner of the prompt 612, or to reject the navigation update, such as by clicking or tapping on a GO BACK button in the bottom left corner of the prompt 612. The user input may be provided via any suitable input device, including but not limited to touchscreens, touchpads, buttons, switches, mice, keyboards, keypads, joysticks, and scroll wheels.

In some aspects, the prompt 612 may be displayed at the end of a workflow or series of operations for registering the intraoperative image space with the sensor space (not shown for simplicity). In some implementations, clicking on the YES, UPDATE button may cause the GUI 601 to display the updated navigation feature 622 in the UPDATED NAVIGATION view. In some implementations, clicking on the GO BACK button may cause the GUI 601 to display the navigation feature 602 in the PRE NAVIGATION UPDATE view. In some other implementations, clicking on the GO BACK button may cause the GUI 601 to return to the workflow for registering the intraoperative image space with the sensor space.

With reference to FIG. 6C, the GUI 601 is shown to display the updated navigation feature 622 in the UPDATED NAVIGATION view. For example, the toggle feature 603 is currently active and toggled to an UPDATED NAVIGATION state. While operating in the UPDATED NAVIGATION state, the navigation feature 622 may display an updated spatial relationship between the instrument 605 and the target 604 based on corrected sensor data. With reference for example to FIG. 5, the user interface 550 may apply the correction 506 to real-time sensor data 501 when generating the 3D model for display in the updated navigation feature 622.

As described with reference to FIG. 5, the correction determination component 540 may determine multiple corrections 506 over time based on updated intraoperative image data. In some implementations, the user interface 550 may apply the most recent correction 506 to the sensor data 501 when generating the 3D model for display in the updated navigation feature 622. In some other implementations, the user interface 550 may further provide the user with an option (such as on the GUI 601) to select one of the corrections 506 to be applied to the sensor data 501 when generating the 3D model for display in the updated navigation feature 622. For example, the toggle feature 603 may include an option to toggle between applying the current correction 506 to the sensor data 501 (based on the most recently acquired intraoperative image data) or applying an older correction to the sensor data 501 (based on intraoperative image data acquired from a previous scan of the anatomy).

With reference to FIG. 6D, the GUI 601 is shown to display the navigation feature 602 in the PRE NAVIGATION UPDATE view. For example, the toggle feature 603 is currently active and toggled to a PRE NAVIGATION UPDATE state. While operating in the PRE NAVIGATION UPDATE state, the navigation feature 602 may display the spatial relationship between the instrument 605 and the target 604 based on uncorrected sensor data. With reference for example to FIG. 5, the user interface 550 may not apply the correction 506 to the real-time sensor data 501 when generating the 3D model for display in the navigation feature 602.

In some aspects, a user may interact with the toggle feature 603 at any time (after the toggle feature 603 becomes active) to toggle between the PRE NAVIGATION UPDATE view (such as shown in FIG. 6C) and the UPDATED NAVIGATION view (such as shown in FIG. 6D). The user input may be provided via any suitable input device, including but not limited to touchscreens, touchpads, buttons, switches, mice, keyboards, keypads, joysticks, and scroll wheels. Accordingly, the toggle feature 603 allows the user to dynamically view the current or real-time spatial relationship between the instrument 605 and the target 604 with or without the correction associated with the intraoperative image space (and without re-initializing navigation, which can be a cumbersome procedure).

Any specific text, fonts, shapes, buttons, icons, or other graphical features shown in the GUI 601 are merely for purposes of illustration and may be configured differently (or user-configurable) in actual implementations. For example, the UPDATED NAVIGATION button may be replaced with different variations of the same or similar textual content (such as “corrected navigation,” “apply correction,” or “redo correction,” among other examples). Similarly, the PRE NAVIGATION UPDATE button also may be replaced with different variations of the same or similar textual content (such as “uncorrected navigation,” “remove correction,” or “undo correction,” among other examples). Various aspects of the GUI 601 also may be customized to user preferences. Example suitable customization options may include, among other examples, changing the number or locations of the views relative to the GUI 601, changing the relative dimensions of one or more views, adjusting various rendering parameters (such as color, opacity, or intensity of various features), or changing the colors of text or highlights in the GUI 601.

In the example of FIGS. 6A-6D, the navigation feature 602 is shown to display a single anatomical model at any given time (such as with or without navigation updates). In some implementations, the navigation feature 602 may concurrently display multiple anatomical models. For example, the navigation feature 602 may display a first anatomical model without navigation updates (such as shown in the example of FIGS. 6A and 6D) and a second anatomical model with the navigation updates (such as shown in the example of FIG. 6C). In some other implementations, a single anatomical model may be used to display the spatial relationship between the instrument 605 and the target 604 with and without the navigation updates. For example, the navigation feature 602 may use the uncorrected sensor data to display the spatial relationship between the instrument 605 and the target 604 in the anatomical model and may use the corrected sensor data to display the updated spatial relationship between the instrument 605 and the target 604 as an overlay on the anatomical model.

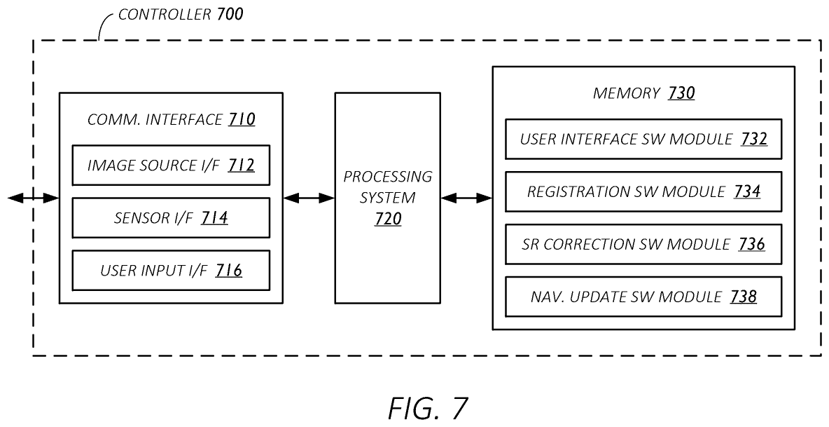

FIG. 7 shows a block diagram of an example controller 700 for a medical system, according to some implementations. In some implementations, the controller 700 may be one example of the navigation system 500 of FIG. 5. More specifically, the controller 700 is configured to generate a graphical user interface (GUI) that depicts or otherwise indicates a spatial relationship between a medical instrument and a target within an anatomy.

The controller 700 includes a communication interface 710, a processing system 720, and a memory 730. The communication interface 710 is configured to communicate with one or more components of the medical system. More specifically, the communication interface 710 includes an image source interface (I/F) 712 for communicating with one or more image sources (such as the CT imaging system 310 and/or the fluoroscopy imaging system 312 of FIG. 3), a sensor interface (I/F) 714 for communicating with one or more sensors (such as the EM sensor system 306 of FIG. 3), and a user input interface (I/F) 716 for communicating with one or more user input devices (such as any of the user input controls 214 and/or the I/O components 240 of FIG. 2). In some implementations, the image source I/F 712 may receive image data captured by an imaging system external to the anatomy while the instrument is disposed within the anatomy. In some implementations, the sensor I/F 714 may receive sensor data via a sensor disposed on the instrument. In some implementations, the user input I/F 714 may receive user input associated with the GUI.

The memory 730 may include a non-transitory computer-readable medium (including one or more nonvolatile memory elements, such as EPROM, EEPROM, Flash memory, or a hard drive, among other examples) that may store the following software (SW) modules: a user interface SW module 732 to generate the GUI, including a first navigation feature depicting a spatial relationship between the instrument and the target; a registration SW module 734 to determine a mapping between a first coordinate space associated with the imaging system and a second coordinate space associated with the sensor based at least in part on the image data and the sensor data; a spatial relationship (SR) correction SW module 736 to determine an updated spatial relationship between the instrument and the target based at least in part on the mapping between the first coordinate space and the second coordinate space; and a navigation update SW module 738 to display, on the GUI, the first navigation feature or a second navigation feature depicting the updated spatial relationship between the instrument and the target based on the received user input. Each of the software modules 732-738 includes instructions that, when executed by the processing system 720, causes the controller 700 to perform the corresponding functions.

The processing system 720 may include any suitable one or more processors capable of executing scripts or instructions of one or more software programs stored in the controller 700 (such as in the memory 730). For example, the processing system 720 may execute the user interface SW module 732 to generate the GUI, including a first navigation feature depicting a spatial relationship between the instrument and the target. The processing system 720 also may execute the registration SW module 734 to determine a mapping between a first coordinate space associated with the imaging system and a second coordinate space associated with the sensor based at least in part on the image data and the sensor data. The processing system 720 may execute the SR correction SW module 736 to determine an updated spatial relationship between the instrument and the target based at least in part on the mapping between the first coordinate space and the second coordinate space. The processing system 720 may further execute the navigation update SW module 738 to display, on the GUI, the first navigation feature or a second navigation feature depicting the updated spatial relationship between the instrument and the target based on the received user input.

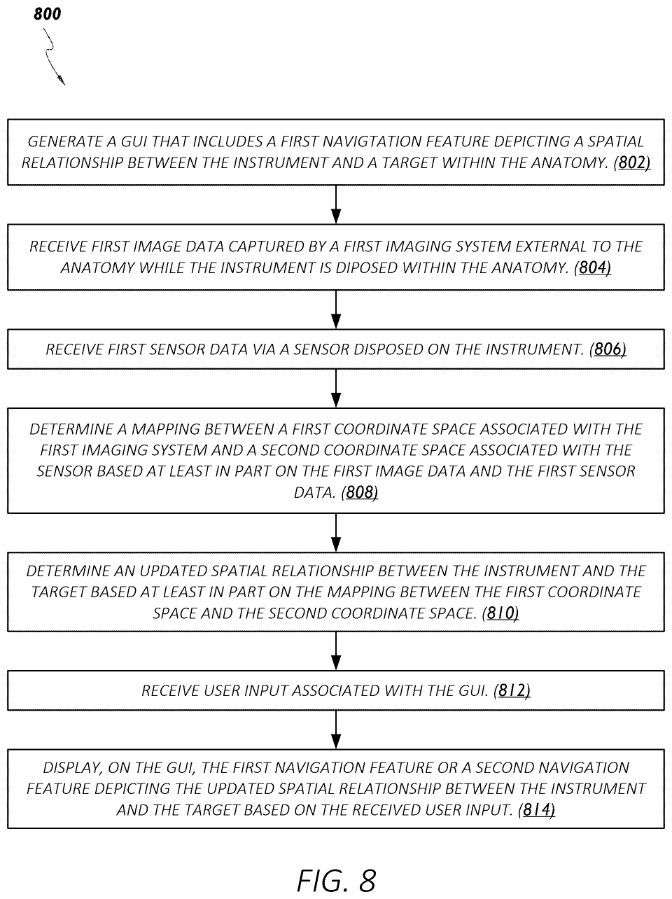

FIG. 8 shows an illustrative flowchart depicting an example operation 800 for navigating an instrument within an anatomy, according to some implementations. In some implementations, the example operation 800 may be performed by a controller for a medical system such as the controller 700 of FIG. 7 or the navigation system 500 of FIG. 5.

The controller may generate a GUI that includes a first navigation feature depicting a spatial relationship between the instrument and a target within the anatomy (802). The controller may receive first image data captured by a first imaging system external to the anatomy while the instrument is disposed within the anatomy (804). In some implementations, the first imaging system may be a cone beam computed tomography (CBCT) system. The controller may receive first sensor data via a sensor disposed on the instrument (806). The controller may determine a mapping between a first coordinate space associated with the first imaging system and a second coordinate space associated with the sensor based at least in part on the first image data and the first sensor data (808). The controller may determine an updated spatial relationship between the instrument and the target based at least in part on the mapping between the first coordinate space and the second coordinate space (810). The controller may receive user input associated with the GUI (812). The controller may further display, on the GUI, the first navigation feature or a second navigation feature depicting the updated spatial relationship between the instrument and the target based on the received user input (814).

In some aspects, the display of the second navigation feature may include determining a correction associated with the updated spatial relationship and applying the correction to the spatial relationship depicted by the first navigation feature. In some implementations, the displaying of the first navigation feature or the second navigation feature may include displaying a toggle feature indicating which of the first or second navigation features is displayed on the GUI and toggling between the first navigation feature and the second navigation feature for display on the GUI responsive to the user input. In some other implementations, the displaying of the first navigation feature or the second navigation feature may include displaying the first navigation feature and the second navigation feature concurrently in the GUI.

In some aspects, the generating of the GUI may include receiving second image data captured by a second imaging system external to the anatomy while the instrument is not disposed within the anatomy; receiving second sensor data via the sensor disposed on the instrument; determining a mapping between the second coordinate space and a third coordinate space associated with the second imaging system based at least in part on the second image data and the second sensor data; and determining the spatial relationship between the instrument and the target based at least in part on the mapping between the second coordinate space and the third coordinate space. In some implementations, the second imaging system may be a computed tomography (CT) system.

In some aspects, the first navigation feature may include an anatomical model having positions of the instrument and the target overlaid thereon based on their spatial relationship. In some implementations, the second navigation feature may include the anatomical model having positions of the instrument and the target overlaid thereon based on their updated spatial relationship. In some aspects, the controller may further receive second image data via the first imaging system subsequent to receiving the first image data and update the mapping between the first coordinate space and the second coordinate space based at least in part on the second image data.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method of navigating an instrument within an anatomy, comprising:
generating a graphical user interface (GUI) that includes a first navigation feature depicting a spatial relationship between the instrument and a target within the anatomy;
receiving first image data captured by a first imaging system external to the anatomy while the instrument is disposed within the anatomy;
receiving first sensor data via a sensor disposed on the instrument;
determining a mapping between a first coordinate space associated with the first imaging system and a second coordinate space associated with the sensor based at least in part on the first image data and the first sensor data;
determining an updated spatial relationship between the instrument and the target based at least in part on the mapping between the first coordinate space and the second coordinate space;
receiving user input associated with the GUI; and
displaying, on the GUI, the first navigation feature or a second navigation feature depicting the updated spatial relationship between the instrument and the target based on the received user input.

2. The method of claim 1, wherein the first imaging system is a cone beam computed tomography (CBCT) system.

3. The method of claim 1, wherein the displaying of the second navigation feature comprises:
determining a correction associated with the updated spatial relationship; and
applying the correction to the spatial relationship depicted by the first navigation feature.

4. The method of claim 1, wherein the displaying of the first navigation feature or the second navigation feature comprises:
displaying a toggle feature indicating which of the first or second navigation features is displayed on the GUI; and
toggling between the first navigation feature and the second navigation feature for display on the GUI responsive to the user input.

5. The method of claim 1, wherein the generating of the GUI comprises:
receiving second image data captured by a second imaging system external to the anatomy while the instrument is not disposed within the anatomy;
receiving second sensor data via the sensor disposed on the instrument;
determining a mapping between the second coordinate space and a third coordinate space associated with the second imaging system based at least in part on the second image data and the second sensor data; and
determining the spatial relationship between the instrument and the target based at least in part on the mapping between the second coordinate space and the third coordinate space.

6. The method of claim 5, wherein the second imaging system is a computed tomography (CT) system.

7. The method of claim 1, further comprising:
receiving second image data via the first imaging system subsequent to receiving the first image data; and
updating the mapping between the first coordinate space and the second coordinate space based at least in part on the second image data.

8. The method of claim 1, wherein the first navigation feature includes an anatomical model having positions of the instrument and the target overlaid thereon based on their spatial relationship.

9. The method of claim 8, wherein the second navigation feature includes the anatomical model having positions of the instrument and the target overlaid thereon based on their updated spatial relationship.

10. The method of claim 1, wherein the displaying of the first navigation feature or the second navigation feature comprises:
displaying the first navigation feature and the second navigation feature concurrently in the GUI.

11. A controller for a medical system, comprising:
a processing system;
a memory storing instructions that, when executed by the processing system, cause the controller to:
generate a graphical user interface (GUI) that includes a first navigation feature depicting a spatial relationship between an instrument and a target within an anatomy;
receive first image data captured by a first imaging system external to the anatomy while the instrument is disposed within the anatomy;
receive first sensor data via a sensor disposed on the instrument;
determine a mapping between a first coordinate space associated with the first imaging system and a second coordinate space associated with the sensor based at least in part on the first image data and the first sensor data;
determine an updated spatial relationship between the instrument and the target based at least in part on the mapping between the first coordinate space and the second coordinate space;
receive user input associated with the GUI; and
display, on the GUI, the first navigation feature or a second navigation feature depicting the updated spatial relationship between the instrument and the target based on the received user input.

12. The controller of claim 11, wherein the first imaging system is a cone beam computed tomography (CBCT) system.

13. The controller of claim 11, wherein the displaying of the second navigation feature comprises:
determining a correction associated with the updated spatial relationship; and
applying the correction to the spatial relationship depicted by the first navigation feature.

14. The controller of claim 11, wherein the displaying of the first navigation feature or the second navigation feature comprises:
displaying a toggle feature indicating which of the first or second navigation features is displayed on the GUI; and
toggling between the first navigation feature and the second navigation feature for display on the GUI responsive to the user input.

15. The controller of claim 11, wherein the generating of the GUI comprises:
receiving second image data captured by a second imaging system external to the anatomy while the instrument is not disposed within the anatomy;
receiving second sensor data via the sensor disposed on the instrument;
determining a mapping between the second coordinate space and a third coordinate space associated with the second imaging system based at least in part on the second image data and the second sensor data; and
determining the spatial relationship between the instrument and the target based at least in part on the mapping between the second coordinate space and the third coordinate space.

16. The controller of claim 15, wherein the second imaging system is a computed tomography (CT) system.

17. The controller of claim 11, wherein execution of the instructions further causes the controller to:
receive second image data via the first imaging system subsequent to receiving the first image data; and
update the mapping between the first coordinate space and the second coordinate space based at least in part on the second image data.

18. The controller of claim 11, wherein the first navigation feature includes an anatomical model having positions of the instrument and the target overlaid thereon based on their spatial relationship.

19. The controller of claim 18, wherein the second navigation feature includes the anatomical model having positions of the instrument and the target overlaid thereon based on their updated spatial relationship.

20. The controller of claim 11, wherein the displaying of the first navigation feature or the second navigation feature comprises:
displaying the first navigation feature and the second navigation feature concurrently in the GUI.
